(19) United States
(12) Patent Application Publication
(10) Pub. No.: US 2025/0022565 A1
(43) Pub. Date: Jan. 16, 2025

(54) ELECTRONIC DEVICE FOR PREDICTING DRUG-DRUG INTERACTIONS AND CONTROL METHOD THEREFOR

(71) Applicant: GWANGJU INSTITUTE OF SCIENCE AND TECHNOLOGY, Gwangju (KR)

(72) Inventors: Hojung NAM, Gwangju (KR); Eunyoung KIM, Gwangju (KR)

(21) Appl. No.: 18/713,452

(22) PCT Filed: Mar. 29, 2022
(86) PCT No.: PCT/KR22/04438

§ 371 Date: May 24, 2024

(30) Foreign Application Priority Data
Nov. 26, 2021 (KR) 10-2021-0165331

(51) Int. Cl. G16H 20/10; G16B 25/10; G16B 40/00; G16H 50/20

(52) U.S. Cl. CPC G16H 20/10; G16B 25/10; G16B 40/00; G16H 50/20

(57) ABSTRACT

An electronic device for predicting drug-drug interactions (DDI), according to an embodiment of the present invention, comprises a processor which identifies the gene expression information of a drug by using drug structures information and drug properties information, and identifies information on DDIs according to simultaneous administration of a first drug and a second drug by using the gene expression information and information on side effects according to drug combination.

### TECHNICAL FIELD

The present disclosure relates to an electronic device for predicting drug interaction and a control method thereof.

### BACKGROUND ART

Due to polypharmacy, the number of patients co-administering multiple drugs is increasing. When the multiple drugs are co-administered, the expected response to each drug may be changed, and in some cases, problems such as unexpected adverse side effects or deterioration of therapeutic efficacy may occur.

In this way, when two or more drugs are taken at the same time, the effect changes are called adverse drug-drug Interactions (DDIs). Proactively identifying potential DDIs is important for patients and the elderly who are prime targets for polypharmacy.

The DDIs is generally discovered through experiments, but in vitro and in vivo identification of DDIs not only increases time and cost, but is also difficult to implement due to patient safety and ethical issues.

Currently, the DDIs may be predicted by similarity-based and network-based approaches such as using data sets retrieved from past research, electronic health records and social media.

However, since most studies have ignored model interpretability and only used features derived from drug compounds, the model cannot capture the characteristics of the DDIs mechanism derived from drug therapy. Furthermore, predicting the interactions of new compounds not used in training is still one of the challenges to be solved.

### DISCLOSURE

### Technical Problem

An object of the present disclosure is to provide an electronic device for performing an interpretable prediction of drug-drug interactions and a control method thereof.

An object of the present disclosure is to provide an electronic device predicting the potential drug-drug interactions even for new drugs not used in training and a control method thereof.

### Technical Solution

In an electronic device for predicting DDIs according to an embodiment of the present disclosure, the electronic device includes: a processor configured to identify gene expression information of a drug using drug structures information and drug properties information, and to identify information on drug-drug interactions according to co-administration of a first drug and a second drug using the gene expression information and information on side effects according to drug combination.

The processor may identify the gene expression information based on a first model learned to calculate the gene expression information according to the drug structures information and the drug properties information.

The processor may identify the information on drug-drug interactions based on a second model learned to calculate the information on drug-drug interactions according to the gene expression information and the information on side effects.

The second model may be a model learned to calculate the information on drug-drug interactions by assigning a weight to the gene expression information.

The processor may identify the information on drug-drug interactions by calculating a distance between the gene expression information of the first drug and the gene expression information of the second drug in a side-effect dimension based on the second model.

The information on the drug-drug interactions may include information on presence of the side effects and the degree of the side effects according to the co-administration of the first drug and the second drug.

In a control method of an electronic device for predicting DDIs according to an embodiment of the present disclosure, the electronic device includes: identifying gene expression information of a drug using drug structures information and drug properties information; and identifying information on drug-drug interactions according to co-administration of a first drug and a second drug using the gene expression information and information on side effects according to drug combination.

The identifying of the gene expression information of a drug may include identifying the gene expression information based on a first model learned to calculate the gene expression information according to the drug structures information and the drug properties information.

The identifying the information on drug-drug interactions may include identifying the information on drug-drug interactions based on a second model learned to calculate the information on drug-drug interactions according to the gene expression information and the information on side effects.

### Advantageous Effects

According to an embodiment of the present disclosure, the side effects that may occur when co-administrating two or more drugs may be predicted more quickly and easily through a trained model.

According to an embodiment of the present disclosure, the possibility of interaction between new drugs or compounds may be predicted more reliably.

According to an embodiment of the present disclosure, a model having a high DDIs prediction accuracy may be provided while providing model interpretability by reflecting gating mechanisms and side effects information.

### DESCRIPTION OF DRAWINGS

FIG. 1 is a diagram illustrating drug-drug interactions.

FIG. 2 is a block diagram illustrating a configuration of an electronic device according to an embodiment of the present disclosure.

FIG. 3 is a flowchart illustrating operations of an electronic device according to an embodiment of the present disclosure.

FIG. 4 is a view illustrating a learning of a first model according to an embodiment of the present disclosure.

FIG. 5 is a diagram illustrating learning a second model according to an embodiment of the present disclosure.

FIG. 6 is a diagram illustrating a score calculation module of a second model according to an embodiment of the present disclosure.

### MODES OF THE INVENTION

Hereinafter, preferred embodiments according to the present disclosure will be described in detail with reference to the accompanying drawings. The detailed description that will be set forth below in conjunction with the accompanying drawings is intended to describe exemplary embodiments of the invention and is not intended to represent the only embodiments in which the invention may be practiced. In the drawings, parts irrelevant to the description may be omitted to clearly describe the present disclosure, and the same reference numerals may be used for the same or similar components throughout the specification. In addition, in the embodiments of the present disclosure, the terms including an ordinal number such as first, second, etc., are used only for the purpose of distinguishing one element from another element, and the singular expression includes the plural expression unless the context clearly dictates otherwise.

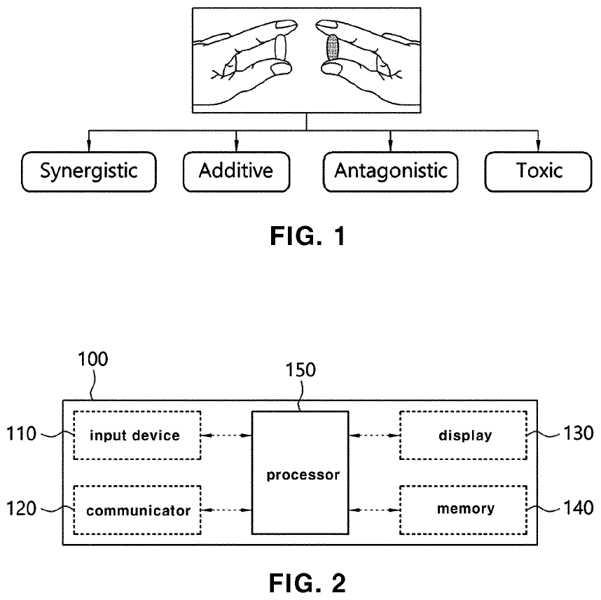

FIG. 1 is a diagram illustrating drug-drug interactions according to an embodiment of the present disclosure.

When two or more drugs are co-administered, drug-drug interactions (DDIs may be used interchangeably as follows) appear in positive directions with a synergistic effect such as synergetic and additive effects, but also in negative directions such as antagonistic or toxic side effects.

The drug-drug interactions are more frequent as the number of drugs increases, and are therefore more likely to occur in patients or the elderly taking a lot of drugs. Experiments for a drug-pair in all cases are impossible in terms of time or cost, and side effects are relatively low in frequency and may occur in various ways, so it is difficult to detect them through experiments.

In the following description, the electronic device 100 that reflects gene expression information of a drug, performs high accuracy prediction of DDIs through model interpretability of DDIs, and predicts potential DDIs even for new drugs not used in training will be described in detail.

FIG. 2 is a block diagram illustrating a configuration of an electronic device according to an embodiment of the present disclosure. The electronic device 100, which is an apparatus for predicting a drug-drug interactions according to an embodiment of the present disclosure, includes an input device 110, a communicator 120, a display 130, a memory 140, and a processor 150.

The input device 110 generates input data in response to a user input of the electronic device 100. The input device 110 includes at least one input means. The input device 110 includes at least one input means. The input device 110 may include a key board, a key pad, a dome switch, a touch panel, a touch key, a mouse, a menu button, and the like.

The communicator 120 communicates with an external device (not shown), for example, a server that provides information necessary to predict information on drug-drug interactions that appear when two or more drugs are co-administered, such as drug structures information or drug properties information. To this end, the communicator 120 may perform communication such as 5th generation communication (5G), long term evolution-advanced (LTE-A), long term evolution (LTE), wireless fidelity (Wi-Fi).

The display 130 displays display data according to an operation of the electronic device 100. The display 130 includes a liquid crystal display (LCD), a light emitting diode (LED) display, an organic light emitting diode (OLED) display, a micro electro mechanical systems (MEMS) display, and an electronic paper display. The display 130 may be combined to the input device 110 and may be implemented as a touch screen.

The memory 140 stores operation programs of the electronic device 100. The memory 140 includes a non-volatile attribute storage capable of storing data (information) regardless of whether or not power is provided, and a volatile attribute memory in which data to be processed by the processor 150 is loaded and data cannot be stored unless power is supplied. The storage includes a flash memory, a hard-disc drive (HDD), a solid-state drive (SSD), a read only memory (ROM), and the like, and the memory includes a buffer, a random access memory (RAM), and the like.

The memory 140 may store drug structures information and drug properties information, or may store gene expression information of a drug identified by the processor 150. In addition, the memory 140 may store information on a first model learned to calculate the gene expression information according to the drug structures information and drug properties information, and information on a second model learned to calculate information on drug-drug interactions that appears when two or more drugs are administered according to the gene expression information.

The processor 150 may execute software such as a program to control at least one other component (e.g., hardware or software component) of the electronic device 100 and may perform various data processing or calculations.

Meanwhile, the processor 150 may perform at least a part of data analysis, processing, and resulting information generation using at least one of machine learning, neural network, or deep learning algorithms as rule-based or artificial intelligence algorithms to identify gene expression information of a drug using drug structures information and drug properties information, and to identify information on drug-drug interactions according to the simultaneous administration of the first drug and the second drug using information on side effects according to gene expression information and information on side effects according to drug combination.

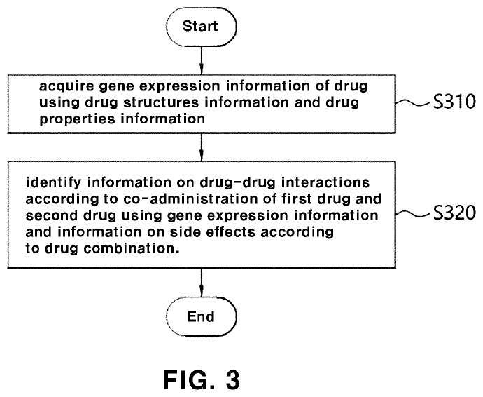

FIG. 3 is a flowchart illustrating operations of an electronic device according to an embodiment of the present disclosure.

According to an embodiment of the present disclosure, the processor 150 identifies gene expression information of a drug by using the drug structures information and drug properties information (S310).

The drug structures information and drug properties information are factors that are mainly used to express drugs. The drug structures information may be expressed as a binary vector using a hashing function by digitizing a structure within a specific radius for each atom in a compound using a molecular structure fingerprint, for example, Morgan Fingerprint.

The drug properties information means information that quantitatively describes physical and chemical information on a molecule with respect to a molecular structure of the compound.

The processor 150 may perform a preprocessing operation or select information to be used to use some property information required to identify gene expression information among numerous property information. For example, the processor 150 may obtain data about the compound from a known database such as a Mordred descriptor, and then select only 100 significant property information using a random forest model. Alternatively, the processor 150 may receive the property information selected from the outside through this process, and is not limited to either one.

According to an embodiment of the present disclosure, it is assumed to perform a prediction on the drug interaction in the case of the simultaneous administration of the first drug and the second drug. The processor 150 may identify the gene expression information of the first drug using the drug structures information and the drug properties information of the first drug, and may identify the gene expression information of the second drug using the drug structures information and the drug properties information of the second drug.

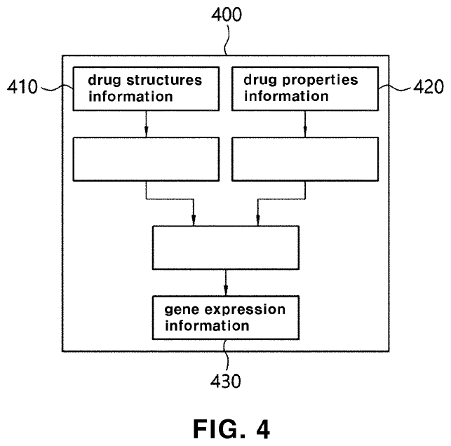

At this time, the processor 150 may identify the gene expression information of the first drug and the second drug based on a first model trained to calculate the gene expression information according to the drug structures information and the drug properties information. Details of the first model will be described in FIG. 4.

According to an embodiment of the present disclosure, the processor 150 identifies information on interactions between drugs according to the simultaneous administration of the first drug and the second drug using information on side effects according to the gene expression information and combination of the drug (S320).

In an embodiment of the present disclosure, the information on side effects according to the drug combination includes side effects information known or tested to occur when the two specific drugs are co-administered.

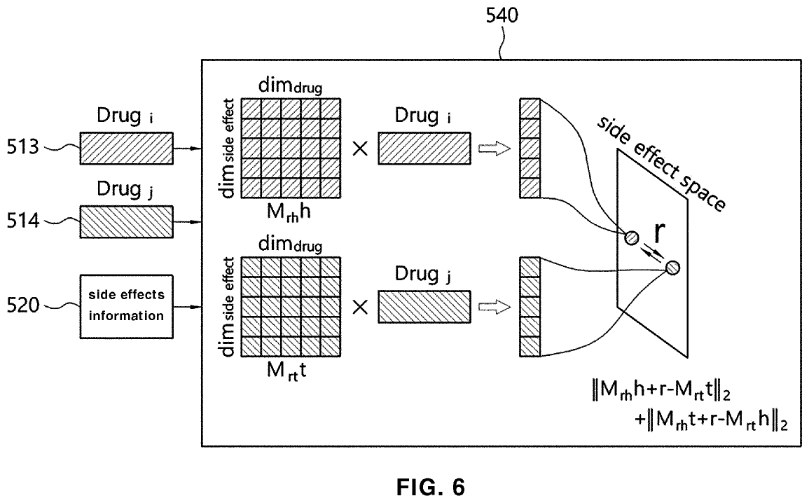

In an embodiment of the present disclosure, the information on the interactions between drugs includes information on presence of the side effects and the degree of the side effects according to the simultaneous administration of the first drug and the second drug. More specifically, the processor 150 identifies information on presence of the side effects and the degree of the side effects by calculating the distance between the gene expression information of two drugs in side-effect dimension. This is described in detail in FIG. 6.

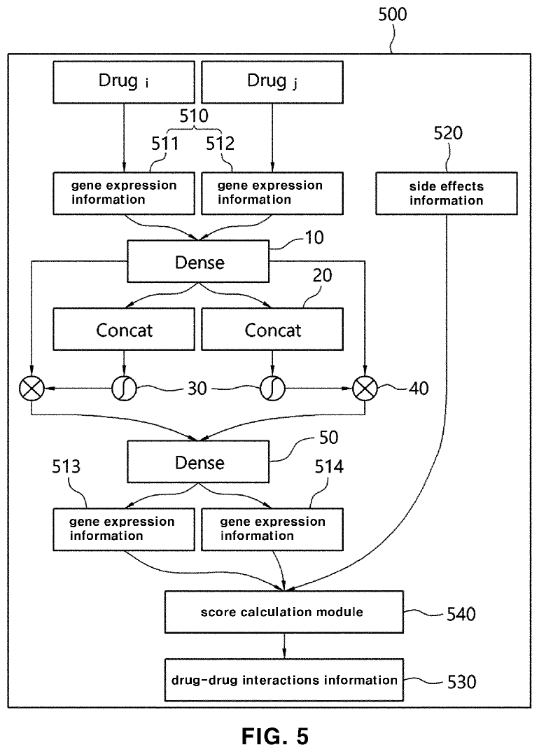

At this time, the processor 150 may identify the information on interactions between drugs based on a second model trained to perform the calculation of the information on the drug-drug interactions that appears when the first and second drugs are simultaneously administered according to the gene expression information. Details of the second model will be described in FIG. 5.

According to an embodiment of the present disclosure, the risk of administering drugs may be reduced by predicting a drug-pair that may cause side effects and what side effects may occur when two or more drugs are administered.

FIG. 4 is a view illustrating a learning of a first model 400 according to an embodiment of the present disclosure.

In an embodiment of the present disclosure, the first model 400 trained to calculate a gene expression information 430 according to a drug structures information 410 and a drug properties information 420 will be described in detail. At this time, the processor 150 may learn the first model 400 or receive, store and use the first model 400, which has been previously learned, from the outside, but is not limited to either one. Hereinafter, operations in the case where the processor 150 learns the first model 400 will be described.

The first model 400 is a model for generating drug-treated gene expression information based on drug information, and uses the structure information 410 and the property information 420 of the drug as input values, and outputs the gene expression information 430 predicted according to the input values as output values.

As the learning data of the first model 400, data obtained by treating multiple cell lines with drugs and measuring gene expression changed after a predetermined time may be used. For example, the standard data set may be a Library of Integrated Network-Based Cellular Signatures (LINCS) L1000 database. The LINCS data includes gene expression information of compounds, cell lines, genes and response signatures.

In this case, before training the neural network using the learning data, the processor 150 may perform a pre-processing operation such as processing the acquired learning data into a preset format, removing noise, and processing the resulting data into an appropriate data form, or select data to be used for learning from among a plurality of learning data.

Therefore, the processor 150 may use LINCS data processed from LINCS raw data as learning data for generating the first model 400. First, the processor 150 extracts gene expression information of 978 directly measured genes for reliability, and since each gene expression information of the compound was taken under various experimental conditions, the processor may select the representative signature of the compound using the “Signature Strength” (SS) value that quantifies the amount of change. Finally, the processor 150 may select organic compounds and obtain gene expression information of 19,156 compounds.

The first model 400 consists of a basic dense layer, and may be learned in a direction of reducing the difference from the known gene expression information.

According to an embodiment of the present disclosure, since it is difficult to obtain gene expression information, which is drug responses information for all drugs, drug-drug interactions may be predicted even for drugs without gene expression information through a model for predicting the gene expression information.

FIG. 5 is a diagram illustrating learning a second model 500 according to an embodiment of the present disclosure.

In an embodiment of the present disclosure, the second model 500 learned to calculate the drug-drug interactions information 530 occurring when two or more drugs are administered in accordance with an information 520 on side effects according to the gene expression information 510 and drug combination will be described in detail. At this time, the processor 150 may learn the second model 500 or receive, store and use the second model 500, which has been previously learned, from the outside, but is not limited to either one. Hereinafter, operations in the case where the processor 150 learns the second model 500 will be described.

The second model 500 is a DDIs prediction model, and uses the gene expression information 510 and the side effects information 520 of the drug as input values, and outputs the drug-drug interactions information 530 predicted according to the input values as output values.

In this case, the gene expression information 510 of the drug may be the gene expression information 430 identified using the structure information of the drug and the property information of the drug as described in step S310 of FIG. 3, or may be the gene expression information 430 identified using the first model 400 as described in FIG. 4, but is not limited to either one.

The second model 500 may use the gene expression information 510 of the drug and the side effects information 520 on the known a drug-pair as the learning data. The side effects information 520 may be, for example, information on polypharmacy side effects retrieved from US FDA's side effects reporting system.

In this case, before training the neural network using the learning data, the processor 150 may perform a pre-processing operation such as processing the acquired learning data into a preset format, removing noise, and processing the resulting data into an appropriate data form, or select data to be used for learning from among a plurality of learning data.

Therefore, the processor 150 may preprocess and use information on polypharmacy side effects retrieved from the US FDA's side effects reporting system as learning data for generating the second model 500.

Since the second model 500 identifies information on drug-drug interactions when two drugs are administered, there are two sets of layers for processing the gene expression information 510. For example, as shown in FIG. 5, a gene expression information 511 of the first drug (Drug i) and a gene expression information 512 of the second drug (Drug j) are used.

Each gene expression information 510 passes through an upper Dense layer 10 in the input layer, and is processed in combination at a Concat layer 20. At this time, the processor 150 uses a gated linear unit (GLU) 30 to represent co-administration effects, which is an effect of two drugs on each other.

The GLU 30 is a gating mechanism that may learn to give a weight to the important gene expression information 510, and uses a sigmoid function to calculate the gene expression information 510 into a value between zero and one.

The GLU 30 reduces the layers combined in the Concat layer 20 to the same dimension as the input layer. Therefore, the dimensions of the two gene expression information 510 may become the same, so that the weights of each layer may be shared.

Afterwards, the value of the calculated gene expression information 510 is multiplied by the weight of each layer through Hadamard product 40 to select important information among the gene expression information 510, thereby controlling the amount of information to be transmitted. At this time, the Hadamard product 40 means an element-wise multiplication of each element of two layers having the same dimension.

More specifically, the GLU 30 is a process of selecting important gene expression information 510 related to the interaction between given drugs. Therefore, the GLU 30 is a gating mechanism developed to control information propagating when passing layers as an output gate with long short-term memory, which may be expressed by Equation (1).

$$GLU ( A , B ) = A ⊗ σ ( B ) \qquad ( 1 )$$

A is the output of the former layer, which is the information to propagate, B is the input of the gate σ, which controls how much of A to use by applying sigmoid non-linearity. With the element-wise multiplication of two parts, it outputs.

The processor 150 reduces the dimensions of the gene expression information 510 passing through the Hadamard product 40 in the lower Dense layer 50. This is to increase the calculation speed and secure the capacity of the memory 140. The lower Dense layer 50 also shares a layer between drugs.

In addition, the processor 150 may output a gene expression information 513 of the first drug (Drug i) and a gene expression information 514 of the second drug (Drug j) of which the weight of each element is adjusted.

According to an embodiment of the present disclosure, the layers between two drugs may be shared, and even if an inputted drug order is changed, the same information 530 on drug-drug interactions may be derived. For example, when input to the first drug (Drug i)-second drug (Drug j) and when input to the second drug (drug j)-first drug (Drug i) may cause a problem in that the output values are different. This means that when the initial weight of each layer is different and the drug order is changed, the output value also changes. On the other hand, the present disclosure may solve this problem by sharing the layers between drugs.

The processor 150 may input the gene expression information 513 and 514 adjusted through the GLU 30 and the side effects information 520 into a score calculation module 540 to identify the drug-drug interactions information 530. More details of the score calculation module 540 will be described in FIG. 6.

According to an embodiment of the present disclosure, the processor 150 dynamically selects the gene expression information using the gating mechanism that reflects the co-administration effects on the gene expression information and learns the second model 500.

According to an embodiment of the present disclosure, it is possible to provide a model having high DDIs prediction accuracy while providing model interpretability by reflecting the gating mechanism and side effects information.

FIG. 6 is a diagram illustrating a score calculation module 540 according to an embodiment of the present disclosure.

According to an embodiment of the present disclosure, the processor 150 may calculate a side-effect score by calculating a distance in a side-effect space between the gene expression information of drugs adjusted through the gating mechanism of the second model 500 described above with reference to FIG. 5.

The gene expression information 513 of the first drug (Drug i) and the gene expression information 514 of the second drug (Drug j) adjusted by the gating mechanism are projected into the side-effect space via translating embedding of the side effects information 520. The side-effect space represents each side effect, and both head and tail nodes each of which represents the start and end points connected by a relation vector are given information on the drug.

At this time, the processor 150 may apply a margin-based loss function to train the score calculation module 540 so that the drug-pair showing side effects included in the side effects information 520 are positioned closely, and if not, farther away. The processor 150 may calculate a side effect score for the corresponding drug-pair for each side effect included in the side effects information 520.

The score of drug-pair, for the first drug di, and the second drug dj, and given side-effect r may be calculated by Equation (2).

$$S ( {d}_{i} , {d}_{j} , r ) = {‖ {M}_{rh} h + r - {M}_{rt} t ‖}_{2} + {‖ {M}_{rh} t + r - {M}_{rt} h ‖}_{2} \qquad ( 2 )$$

Here, h and t are embedding vectors of a drug, r is an embedding vector of a relation, and Mrh and Mrt are mapping matrices multiplied to h and t to project drug features into the side-effect spaces, respectively. Here, since the drug combination data do not have directionality, the distance is calculated by considering either direction.

In conclusion, if the gating mechanism and the translating embedding of the side effects information are used after the gene expression information of a drug are used, the DDIs prediction accuracy may be improved while providing model interpretability.

1. An electronic device for predicting drug-drug interactions (DDIs), the electronic device comprising: a processor configured to:
identify gene expression information of a drug using drug structures information and drug properties information; and
identify information on drug-drug interactions according to co-administration of a first drug and a second drug using the gene expression information and information on side effects according to drug combination.

2. The electronic device of claim 1, wherein the processor is configured to:
identify the gene expression information based on a first model learned to calculate the gene expression information according to the drug structures information and the drug properties information.

3. The electronic device of claim 1, wherein the processor is configured to:
identify the information on drug-drug interactions based on a second model learned to calculate the information on drug-drug interactions according to the gene expression information and the information on side effects.

4. The electronic device of claim 3, wherein the second model is a model learned to calculate the information on drug-drug interactions by assigning a weight to the gene expression information.

5. The electronic device of claim 3, wherein the processor is configured to:
identify the information on drug-drug interactions by calculating a distance between the gene expression information of the first drug and the gene expression information of the second drug in a side-effect dimension based on the second model.

6. The electronic device of claim 1, wherein the information on the drug-drug interactions comprises information on presence of the side effects and the degree of the side effects according to the co-administration of the first drug and the second drug.

7. A control method of an electronic device for predicting drug-drug interactions (DDIs), the control method comprising:
identifying gene expression information of a drug using drug structures information and drug properties information; and
identifying information on drug-drug interactions according to co-administration of a first drug and a second drug using the gene expression information and information on side effects according to drug combination.

8. The control method of claim 7, wherein the identifying of the gene expression information of drug comprises:
identifying the gene expression information based on a first model learned to calculate the gene expression information according to the drug structures information and the drug properties information.

9. The control method of claim 7, wherein the identifying the information on drug-drug interactions comprising:
identifying the information on drug-drug interactions based on a second model learned to calculate the information on drug-drug interactions according to the gene expression information and the information on side effects.

10. The control method of claim 8, wherein the identifying the information on drug-drug interactions comprising:
identifying the information on drug-drug interactions based on a second model learned to calculate the information on drug-drug interactions according to the gene expression information and the information on side effects.

11. The electronic device of claim 2, wherein the processor is configured to:
identify the information on drug-drug interactions based on a second model learned to calculate the information on drug-drug interactions according to the gene expression information and the information on side effects.

12. The electronic device of claim 11, wherein the second model is a model learned to calculate the information on drug-drug interactions by assigning a weight to the gene expression information.

13. The electronic device of claim 11, wherein the processor is configured to:
identify the information on drug-drug interactions by calculating a distance between the gene expression information of the first drug and the gene expression information of the second drug in a side-effect dimension based on the second model.
